(19) United States
(12) Patent Application Publication
(10) Pub. No.: US 2024/0350685 A1
(43) Pub. Date: Oct. 24, 2024

(54) ELECTRONIC FILTER SYSTEM FOR DISINFECTION OF FLUIDS

(71) Applicant: KAF HEALTH TEKNOLOJI ANONIM SIRKETI, Istanbul (TR)

(72) Inventors: Kubra KARAKOC, Istanbul (TR); Furkan ERUCAN, Istanbul (TR)

(73) Assignee: KAF HEALTH TEKNOLOJI ANONIM SIRKETI, Istanbul (TR)

(21) Appl. No.: 18/684,691

(22) PCT Filed: Jul. 7, 2022
(86) PCT No.: PCT/TR2022/050721

§ 371 Date: Feb. 19, 2024

(30) Foreign Application Priority Data
Aug. 18, 2021 (TR) 2021/013068

(51) Int. Cl. A61L 2/02

(52) U.S. Cl. CPC A61L 2/022; A61L 2209/12; A61L 2209/14

(57) ABSTRACT

An electronic filter system for disinfection of all kinds of fluids which need to be disinfected, particularly including air and water. The electronic filter system is designed to be integrated into fluid ducts and channels. The electronic filter system constitutes a standard system in terms of its fundamental systematics, with proper dimensions for the fluid duct or channel the said system to be integrated. The electronic filter system, when used simultaneously with the flow of a fluid through fluid ducts or channels, will ensure disinfection of the fluid without the need for any long disinfection tunnels which are expensive in terms of both installation and power costs.

### CROSS REFERENCE TO THE RELATED APPLICATIONS

This application is the national phase entry of International Application No. PCT/TR2022/050721, filed on Jul. 7, 2022, which is based upon and claims priority to Turkish Patent Application No. 2021/013068, filed on Aug. 18, 2021, the entire contents of which are incorporated herein by reference.

### TECHNICAL FIELD

The present invention relates to an electronic filter system for disinfection of all kinds of fluids which need to be disinfected, particularly including air and water.

The said system according to the present disclosure is designed to be integrated into fluid ducts and channels. The electronic filter system constitutes a standard system in terms of its fundamental systematics, with proper dimensions far the fluid duct or channel the said system to be integrated.

An objective of the present disclosure is to provide such electronic filter that, simultaneously with the flow of a fluid through fluid ducts or channels, will ensure disinfection of the fluid without the need for any long disinfection tunnels which are expensive in terms of both installation and power costs.

### BACKGROUND

In the field of fluid disinfection, currently, there are mainly conventional physical, chemical and non-contact disinfection methods, as well as the innovative Electronic Filter system of the present disclosure.

Physical disinfection methods include flocculation, coagulation, sedimentation, filtration, and heat treatment techniques. Flocculation, coagulation, sedimentation, and filtration techniques are essentially used for cleaning a fluid out of particles that are quite large with a high molecular weight compared to microorganisms in the fluid. Disinfection in such processes is achieved by, and to the extent of, the removal of the said particles. However, in such systems, which only manage to remove particles up to a certain size, smaller particles remaining in flow continue to harbor micron-sized living and disease-causing organisms, i.e. microorganisms. Therefore, exposing fluids to flocculation, coagulation, sedimentation, and filtration techniques is an inadequate solution to provide people with a safe fluid for human health. A heat treatment technique is based on heating a fluid and keeping it under a high temperature for a long period of time. Keeping a fluid under a high temperature for a long period of time in a continuous system is a costly method which is rarely used.

A chemical disinfection method is based on the principle of neutralizing the target microorganisms in a fluid by means of active substances targeting the molecules in the cell wall, cell membrane and cells of such microorganisms. In disinfection systems, chemical disinfectants are generally used in a combination, because there are no single chemical agent that is able to inactivate all kinds of disease-causing microorganisms. Today, chlorine and other chemicals are widely used in fluid disinfection. However, chemical disinfectants leave residues in fluids they are being used in, form by-products, and eventually move to the human body. A long-term exposure of the human body to these chemical products may trigger the onset of cancer, as well as of neurological diseases such as Alzheimer, or such chemicals per se may result in a variety of diseases. As another problem other than the problem of leaving residues in fluid, in order to obtain an effective level of fluid disinfection with chemical disinfectants, one has to let chemical disinfectants to remain within the fluid for a long time, and for certain types of chemical disinfectants, use such agents in a high dosage. And this necessity gives rise to problems related to supply and disposal of such large quantities of chemicals.

Non-contact disinfection methods include ozone and irradiation technologies. Ozone technology is rarely used for disinfection purposes as it is an expensive method requiring a long-term exposure on the target fluid to be effective and the ozone molecule, i.e. the basis of the said technology, turns into an oxygen molecule in a very short amount of time. In irradiation technology, a conventional Ultraviolet Light is the preferred choice, and the conventional Ultraviolet Light type C (UV-C) is often used. The conventional UV-C light can disinfect microorganisms in a fluid if and only if it is kept in contact with the said fluid far a prolonged period of time, and this requires to use long disinfection lines which are expensive.

Described below are the inventions involving conventional fluid disinfection techniques and thus having the above disadvantages.

TR1998/00400 (Photocatalytic Air Disinfection) describes a filter with an air outlet surface coated with a photocatalytic agent (titanium dioxide—TiO₂) wherein a conventional UV light emitted by a photon source acting on the said photocatalyst-coated filter surface leads to a chemical reaction on the said photocatalyst-coated filter surface when a contaminated air is passed through the said filter, and wherein the said photocatalytic oxidation reaction results in the destruction of at least some of the microorganisms in the said contaminated air. The said UV light consists of photons with wavelengths ranging between 300-400 nm (UV-A and UV-B). A disadvantage of this invention is that the wavelength and exposure time of the UV light used therein are insufficient for a complete disinfection. Photons with a UV-C wavelength can be effective in disinfection. Yet, exposure time should be considered in this case as well. A prolonged exposure time require the installation of long disinfection lines, thus resulting in a high cost. Another disadvantage of this filter is that the photocatalytic agent employed in the said invention, i.e. TiO₂, is likely to be transported via air in the form of nanoparticles in which case it may enter the body through respiration and cannot be eliminated from the body, eventually causing a carcinogenic effect by disrupting the structure of cells within the body.

The subject-matter of the patent no. TR2020/07791 (Air Conditioner Disinfection System) is designed to be installed in living room type and cassette type air conditioners. The said system ensures that the ambient air entering via an air suction opening of the air conditioner to be kept inside the cabinet as compressed is disinfected by means of a conventional UV-C light arrangement in the cabinet while it enters the system, following which the said air be blown as a clean air from the air conditioner right after passing through a dust and particulate filter in the air suction duct. Since this system is designed only as a module for living room type and cassette type air conditioners, its application is limited. The location of this system constitutes a disadvantage for the said system because the air being disinfected in the air intake section may be contaminated again within the air conditioning cycle before it leaves the conditioner. Dust and dirt accumulated inside an air conditioner during idle time will be spread out through the air when the air conditioner is started again, and the machine will start to operate efficiently only after a certain period of operation. Another disadvantage of the system is its low disinfection efficiency which stems from the fact that the conventional UV-C light employed in the system is not able to provide a complete disinfection in a short period of time during a continuous flow.

TR2020/11095 (UV-C Air Disinfection Device far Indoor Units of Ductless Air Conditioning Systems) is also related to a system for ductless air conditioners (living room type or cassette type) as in TR2020/07791. In fact, the abovementioned disadvantages for the subject-matter of the patent no. TR2020/07791 are also present here, meaning that this system includes a conventional UV-C light source for disinfection but it is again located upstream of the respective filtering section, rendering the system incapable to prevent any contamination that might occur within the air conditioner, and as described above, the proposed conventional UV-C light source requires a long exposure time for a complete disinfection.

U.S. Pat. No. 8,252,099B2 discloses an apparatus called Air Filtration Device to be used as a standalone unit, and a method for using the said device as an integrated unit within HVAC or AHU systems. This system consists of a plurality of filters, a fan, and a conventional UV-C light source inside a tubular body with two open ends, one for receiving air and one for exhausting air. The air in the external environment is sucked via a fan in the air inlet end of the system, and then transferred to a multi-stage filter located behind the said fan. After passing through the said filter, the air moves through an area where the conventional UV-C light source is located, and then passes through a multi-stage filter on the air outlet side to be obtained as a purified/disinfected air. Since the conventional UV-C light used in this system does not have the ability to perform an instant disinfection, it has the same disadvantages as the subject-matter of the patent no. TR2020/07791. In fact, two separate multi-stage filters, one of which is on the air inlet side and the other on the air outlet side, are used in order to resolve this problem; however, filters cannot block the disease-causing microorganisms below a certain size and thus do not constitute a solution for the problem arising from the inability of the conventional UV-C light source to provide an instant disinfection, resulting in a poor disinfection performance by the system.

U.S. Pat. No. 8,496,735B2 relates to a method or system for sucking the polluted air from the external environment to pass it through a nanocrystalline metal oxide-coated filter and then returning it to the environment. This invention has the same disadvantage as the subject-matter of the patent no. TR1998/00400, which stems from the use of a nanocrystalline photocatalytic agent, meaning that the photocatalytic agent employed here may also be transported via air in which case it may enter the body through respiration and cannot be eliminated from the body, eventually causing a carcinogenic effect by disrupting the structure of cells within the body.

US20150033942A1 relates to a system for use in residential or commercial HVAC systems, wherein the said system comprises gaseous and particulate matter-arresting filters coated with a photocatalytic agent, as well as a conventional UV light source, a sensor, a detector, a switch, a valve, and a microprocessor. In this system which is called Regenerative Air Purification System, the air taken into the system is measured with gas sensors and detectors to determine whether or not the air quality is at the desired level, and accordingly, the presence of an air at the desired quality level results in an uninterrupted flow of air freely passing through, whereas the presence of a polluted air results in a blockage on the air flow which prevents the air from leaving the system and is maintained until the quality of the contained air reaches the desired level. For current HVAC systems, this system constitutes a complex and costly solution. In addition to the disadvantage mentioned above, i.e. occasional interruptions on air flow, this system has also other problems due to the use of nanocrystalline photocatalytic agents therein, namely the same side effects as in the subject-matter of the patent no. TR1998/00400. Moreover, it has also other disadvantages, such as that the small disease-causing microorganisms below a certain size can neither be arrested by filters nor detected by sensors or detectors, and that the ability of the conventional UV light system to perform an instant disinfection is poor.

In conclusion, the abovementioned shortcomings and the inadequacy of the current practice entail an improvement in the respective technical field. Thus, there is a need for an invention to overcome the described problems.

### SUMMARY

In order to eliminate the aforementioned disadvantages and provide new advantages to the respective technical field, the present disclosure provides an electronic filter that, simultaneously with the flow of a fluid through fluid channels, will ensure disinfection of the fluid without the need for any long disinfection tunnels which are expensive in terms of both installation and power costs. The present disclosure relates to an electronic filter system for disinfection of fluids.

The said system of the present disclosure consists of an array of UV-C light sources driven by a “Pulse-Width Modulation and Direct Current Drive Technique” which is designed according to filter dimensions. The system of the present disclosure provides an effective instant disinfection by eliminating the need for a prolonged exposure time, which is the biggest disadvantage of the UV light technologies in the prior art. The instant disinfection technology “Pulse-Width Modulation and Direct Current Drive Technique” used in the system according to the present disclosure is a product of seven years of R&D studies carried out under the consultancy of professors.

This “Pulse-Width Modulation and Direct Current Drive Technique” used in the system of the present disclosure is a device which has at least one driver module, our own design, generating a high-amplitude, high-frequency pulse signal, and at least one UV-C light source generating a high-intensity light and having the proper specifications required for it to operate in harmony with the signal generated by the driver module. This device works with a DC 5V electrical signal. In the driver module, a DC voltage arriving at an input side of a driver IC is amplified. As a result, a high frequency, high-amplitude pulse signal, generally with a switching frequency of 1.2 MHz, is obtained as the output of the driver IC. By using the driver IC, a circuit providing the maximum brightness is generated with the PWM technique. The generated electrical signal is regulated with a filter circuit consisting of RLC elements. And in order to increase the light intensity, the DC electric charge stored in a capacitor in an output side of the driver circuit is combined with the obtained high-frequency, high-amplitude pulse signal, and discharged towards the UV-C light source within microseconds. Thus, a high-frequency, high-amplitude electrical signal obtained from the driver circuit is transmitted to the UV-C light source, resulting in a high-frequency UV-C light at the maximum brightness.

This means that the photons emitted from the UV-C light source penetrates deeply into the fluid by generally hitting it 1.2×10⁶ times per second, thereby inactivating the target microorganisms within microseconds by disrupting their cell structures.

As a result, photons scattered from the high-frequency high-intensity UV-C light, which generally hits 1.2 million times the unit area of the filter to which it is associated with, disinfect the fluid passing through the filter by neutralizing microorganisms below a certain size, which the filters cannot block, without allowing the said microorganisms to spread out from the filter surface.

Other features of the system of the present disclosure that are superior to the conventional fluid disinfection methods are as follows: it is not radioactive; the emitted intense UV-C light does not cause deformation in a physical structure of a fluid it affects; it does not cause any color change; it does not cause particles to break out from the fluid it affects; it does not contain any chemicals; it does not leave any residues; it does not cause any odor or smoke formation; and it does not release any gas.

The electronic filter system developed for use in disinfection of fluids according to the present disclosure can be produced in different sizes, bigger or smaller.

### BRIEF DESCRIPTION OF THE DRAWINGS

The embodiments of the present disclosure which are summarized above and discussed in more detail below can be better understood by referring to exemplary embodiments of the present disclosure illustrated in the accompanying drawings. It should be noted, however, that the accompanying drawings only describe the typical embodiments of the present disclosure, and thus, are not to be considered as limiting the scope of the present disclosure such that other effective embodiments may also be within the scope of the present disclosure.

FIG. 1 illustrates a top view of the present invention.

FIG. 2 illustrates a side view of the present invention.

FIG. 3 illustrates an overall and perspective view of the present invention.

FIG. 4 illustrates a top view of a circuit used in the present invention.

For ease of understanding, identical reference numerals are used where possible to indicate identical elements in the figures. Figures are not drawn to scale and can be simplified for clarity. It is contemplated that elements and features of an embodiment can be usefully incorporated into other embodiments without the need for further explanation.

### DESCRIPTION OF DETAILS IN DRAWINGS

Described herein are the reference numbers shown in the figures.
1. Driver circuit
   1.1 Driver IC
   1.2 Inductor
   1.3 Resistor
   1.4 First capacitor
   1.5 Second capacitor
   1.6 Third capacitor
2. Light source
3. Tape

### DETAILED DESCRIPTION OF THE EMBODIMENTS

The preferred alternative embodiments of the electronic filter system for disinfection of fluids according to the present disclosure as described in this detailed description are only intended for providing a better understanding of the subject-matter, and should not be construed in any restrictive sense.

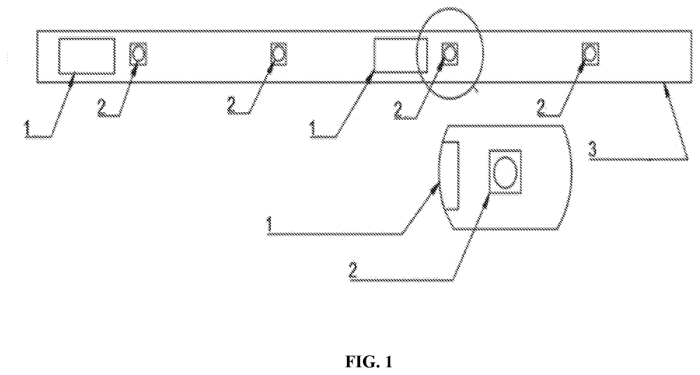

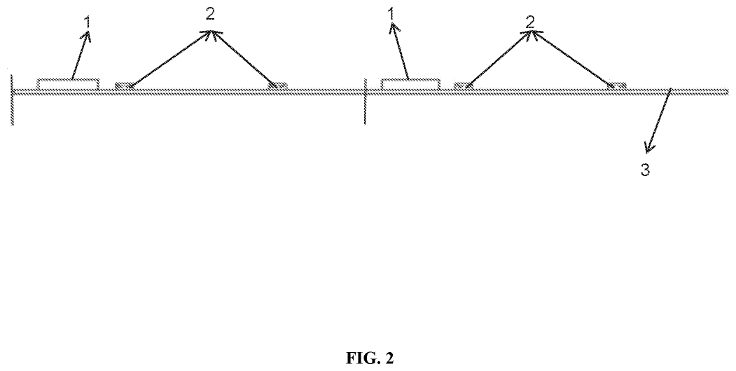

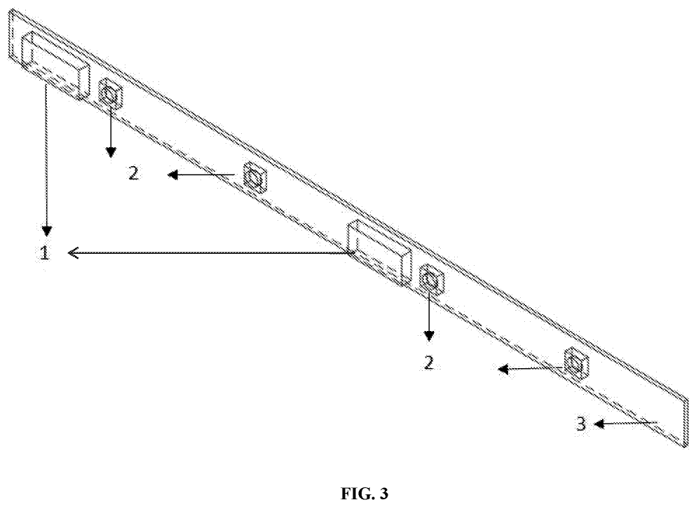

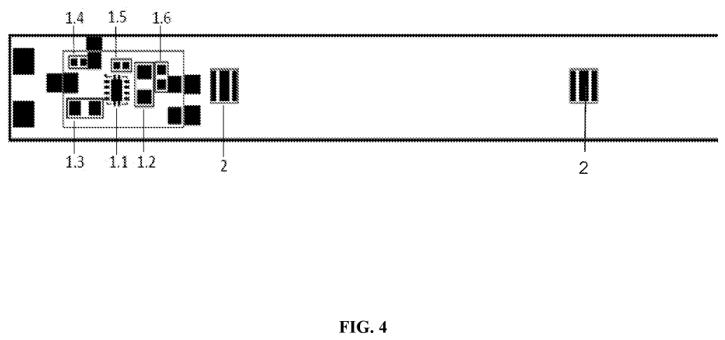

As can be seen in FIGS. 1-4, the present invention is an electronic filter system for use in disinfection of fluids, comprising a driver circuit (1), a light source (2), and a tape (3). In addition, the present invention also includes an “innovative UV-C tape-integrated filter” and an “innovative UV-C tape-integrated electronic filter.”

The driver circuit (1) used in the present invention comprises a driver IC (1.1), an inductor (1.2), a resistor (1.3), a first capacitor (1.4), a second capacitor (1.5), and a third capacitor (1.6).

In a preferred embodiment of the present invention, two tapes (3) are used. In alternative embodiments of the present invention, at least one tape (3) is used.

There are generally two driver circuits (1) and four light sources (2) on each strip (3). Each driver circuit (1) is designed to operate generally with two light sources (2), but this may vary depending upon the disinfection needs. In a preferred embodiment of the present invention, two driver circuits (1) and four light sources (2) are used.

A DC voltage arriving at an power input side of the driver circuit (1) is amplified, and processed by the driver IC (1.1) to convert it into a High-Frequency, High-Amplitude Pulse Signal. The signal processed on the driver IC (1.1) is configured to reach the maximum brightness by applying the PWM technique. By means of the inductor (1.2) in the driver circuit (1) any sudden current changes in this signal are prevented, and by means of the resistor (1.3) the light sources (2) are prevented from drawing an excessive amount of current. The first capacitor (1.4) and the second capacitor (1.5), which are located at an input side, and an output side of the driver circuit (1), respectively, are used for dampening any fluctuations in voltage. The obtained signal is combined with a DC electric charge accumulated in the third capacitor (1.6) located at an input side of the light sources, and is discharged to the light sources (2) within microseconds. Thus, the light sources (2) generate a burst of high-frequency light at a maximum brightness, providing a complete disinfection on the surface it acts on.

The driver IC (1.1) amplifies the input signal to a high-frequency, high-amplitude signal, usually with a switching frequency of 1.2 MHz. The maximum brightness is configured with the PWM technique. However, this value cannot be limited to 1.2 MHz, and any variation in the frequency is also within the scope of the present invention.

As can be seen in FIGS. 1-4, the present invention is characterized in that it comprises the driver circuit (1) which ensures a shorter disinfection time by combining the generated high-frequency, high-amplitude electrical pulse signal with the stored DC electric charge and discharging this combination to the light source (2) to obtain a maximum brightness; as well as at least one light source (2) driven by the power generated in the driver circuit (1); and at least one tape (3) on which both the driver circuit (1) and the light source (2) are positioned.

As can be seen in FIGS. 1-4, the driver circuit (1) used in the present invention comprises the driver IC (1.1) which amplifies the signal at the input side thereof to a high-frequency, high-amplitude signal with a switching frequency of 1.2 MHz; the inductor (1.2), a component of the RLC (Resistor Inductor Capacitor) filter circuit, which dampens any sudden current changes in the output signal and regulates the signal; the resistor (1.3), a component of the RLC filter circuit, which prevents the light sources (2) from drawing an excessive amount of current thanks to resistance force it has; the first capacitor (1.4), a component of the RLC filter circuit, which prevents any fluctuations in voltage at the power input side of the driver circuit (1); the second capacitor (1.5) which prevents any fluctuations in voltage at the output side of the driver IC (1.1); and the third capacitor (1.6) which stores the DC electric charge to increase the light intensity.

The system of the present invention is designed to consist of the innovative UV-C Tape (3) to make the system to have an effect on the outlet surface (rear) of the filter. On this UV-C Tape (3), the Driver Circuit(s) (1) and the UV-C light source(s) 2 are located in a number determined according to the size of the filter. Each Driver Circuit (1) is designed to drive one or more UV-C light source(s) (2).

The DC voltage arriving at the power input side of the Driver Circuit (1) is amplified, and processed by the Driver IC (1.1) to convert it into the High-Frequency, High-Amplitude Pulse Signal. The signal processed on the Driver IC (1.1) is configured to reach the maximum brightness by applying the PWM technique. By means of the Inductor (1.2) in the Driver Circuit (1) any sudden current changes in this signal are prevented, and by means of the Resistor (1.3) the UV-C light source (2) is prevented from drawing an excessive amount of current.

The first capacitor (1.4) and the second capacitor (1.5), which are located at the input side, and the output side of the Driver Circuit (1), respectively, are used for dampening any fluctuations in voltage. The obtained signal is combined with the DC electric charge accumulated in the third capacitor (1.6) located at the input side of the UV-C light source (2), and is discharged to the UV-C light source (2) within microseconds. Thus, the UV-C light source (2) generates a burst of high-frequency light at a maximum brightness, providing a complete disinfection on the fluid it acts on.

The “innovative UV-C tape-integrated filter” is the filter that arrests large pollutants in the fluid. The “innovative UV-C tape-integrated electronic filter” is an innovative UV-C tape-integrated electronic filter that arrests large-sized pollutants in the fluid and then neutralizes the microorganisms remaining in the fluid.

1. An electronic filter system for use in disinfection of fluids, comprising:
a driver circuit which combines a generated high-frequency, high-amplitude electrical pulse signal with a stored DC electric charge and discharges the combination to a light source to obtain a maximum brightness,
at least one light source driven by power generated in the driver circuit, and
at least one tape on which both the driver circuit and the light source are positioned.

2. The electronic filter system according to claim 1, wherein the driver circuit further comprises a driver IC which provides an amplification to a high-frequency, high-amplitude signal.

3. The electronic filter system according to claim 1, wherein the driver circuit further comprises an inductor, a component of the RLC (Resistor Inductor Capacitor) filter circuit, which dampens any sudden current changes in an output signal and regulates the signal.

4. The electronic filter system according to claim 1, wherein the driver circuit further comprises a resistor, a component of the RLC filter circuit, which prevents the light sources from drawing an excessive amount of current.

5. The electronic filter system according to claim 1, wherein the driver circuit further comprises a first capacitor, a component of the RLC filter circuit, which prevents any fluctuations in voltage at the power input side of the driver circuit.

6. The electronic filter system according to claim 1, wherein the driver circuit further comprises a second capacitor which prevents any fluctuations in voltage at an output side of the driver IC.

7. The electronic filter system according to claim 1, wherein the driver circuit further comprises a third capacitor which stores the DC electric charge to increase the light intensity.

8. The electronic filter system according to claim 1, further comprises an filter which arrests large pollutants in the fluid, and an electronic filter which arrests large-sized pollutants in the fluid and then neutralizes the microorganisms remaining in the fluid.
